(19) United States
(12) Patent Application Publication
(10) Pub. No.: US 2003/0219821 A1
(43) Pub. Date: Nov. 27, 2003

(54) Methods for identifying oncogenes and tumor-associated proteins

(76) Inventors: Paul B. Fisher, Scarsdale, NY (US); Neil I. Goldstein, Maplewood, NJ (US)

Correspondence Address: BAKER & BOTTS, 30 ROCKEFELLER PLAZA, NEW YORK, NY, 10112

(21) Appl. No.: 10/139,732

(22) Filed: May 6, 2002

Related U.S. Application Data
(63) Continuation of application No. 09/249,526, filed on Feb. 12, 1999, now abandoned.
(60) Provisional application No. 60/074,580, filed on Feb. 13, 1998.

(51) Int. Cl. G01N 33/53; C07H 21/04; C12P 21/02; C12N 5/06; C07K 16/30

(52) U.S. Cl. 435/7.1; 435/69.1; 435/326; 435/320.1; 530/388.8; 536/23.53

(57) ABSTRACT

This invention provides a method for preparing antibodies directed against a protein associated with a cancer, comprising the steps of:

(a) transfecting non-tumorigenic cells with a cDNA library comprising cDNA molecules derived from mRNA of cancer cells;

(b) masking the transfected cells with antibodies raised against non-tumorigenic cells transfected with a cDNA library derived from normal cells; and

(c) generating antibodies against the masked cells, and thereby generating antibodies directed against a protein associated with a cancer.

This application claims the benefit of copending U.S. Provisional Application Serial No. 60/074,580, filed Feb. 13, 1998, the contents of which are hereby incorporated by reference.

Throughout this application, various references are referred to within parentheses. Disclosures of these publications in their entireties are hereby incorporated by reference into this application to more fully describe the state of the art to which this invention pertains.

### BACKGROUND OF THE INVENTION

The identification of genes and proteins that are specifically expressed by diseased cells, such as tumor cells, is critical for the development of improved therapies and diagnostic procedures. In recent years a number of new techniques have become available for identifying genes and antigens specific to cancers. One such technique is surface-epitope masking (SEM), which involves the selective blocking of surface antigens present on a genetically altered tester cell with high-titer polyclonal antibodies that have been produced against a driver cell (the untransfected parental cell). The masked cells are injected into a suitable host animal (e.g., BALB/c mice) and immune spleen cells taken from the immunized mice are fused with myeloma cells. Using this procedure, hybridomas that secrete monoclonal antibodies that react with cell-surface antigens on the transfected tester cells can be prepared (see Shen et al., J. Natl. Cancer Inst. 86:91-98, 1994). Such antibodies may then be used to identify cell-surface antigens, and genes encoding such antigens. Depending on the source of the transfected DNA, the antigens may be associated with any of a variety of disease states. For example, antigens associated with multidrug-resistant breast carcinoma cells and prostate tumor cells have been identified using this technique (see Shen et al., J. Natl. Cancer Inst. 86:91-98, 1994;WO 96/21671).

Although SEM is useful for identifying certain disease-specific antigens, this technique suffers from certain limitations. For example, only surface antigens may be identified via SEM. Internal antigens, which also have potential utility within diagnostic and/or therapeutic methods are missed entirely. In addition, monoclonal antibodies must be generated for each antigen identified, which substantially increases the time required for antigen identification. Furthermore, surface antigens that may not be specific to the disease state are identified by SEM.

Accordingly, there is a need in the art for improved methods for identifying antigens associated with diseases such as cancer. The present invention fulfills this need and further provides other related advantages.

The present invention relates generally to the identification of genes and proteins associated with cancer or other diseases related to cell growth. The invention is more particularly related to methods for identifying genes and antigens that are expressed at altered levels in cancer cells, as well as cDNA molecules encoding such antigens.

### SUMMARY OF THE INVENTION

This invention provides a method for preparing antibodies directed against a protein associated with a cancer, comprising the steps of:

(a) transfecting non-tumorigenic cells with a cDNA library comprising cDNA molecules derived from mRNA of cancer cells;

(b) masking the transfected cells with antibodies raised against non-tumorigenic cells transfected with a cDNA library derived from normal cells; and

(c) generating antibodies against the masked cells, and thereby generating antibodies directed against a protein associated with a cancer.

This invention provides a method for identifying a cDNA molecule encoding a protein associated with a cancer, comprising the steps of:

(a) transfecting non-tumorigenic cells with a cDNA library comprising cDNA molecules derived from mRNA of cancer cells;

(b) masking the transfected cells with antibodies raised against non-tumorigenic cells transfected with a cDNA library derived form normal cells;

(c) Generating monoclonal antibodies against antigens present on the surface of the masked cells;

(d) preparing a phage display library comprising phage particles that display single chain Fv and/or Fab fragments that bind to antigens present on the surface of the masked cells; and

(e) identifying a cDNA molecule present within the cDNA library derived from cancer cells, wherein the cDNA molecule encodes an antigen that binds to a single chain Fv or Fab fragment present in the phage display library, and therefrom identifying a cDNA molecule encoding a protein associated with a cancer.

This invention provides a method for identifying a cDNA molecule encoding a tumor-associated protein, comprising the steps of:

(a) transfecting non-tumorigenic cells with a cDNA library derived from tumor cells;

(b) injecting the transfected cells into an immunocompromised animal;

(c) explanting any tumors that develop in the animal;

(d) isolating cells that grow from the explanted tumors; and

(e) identifying a cDNA molecule expressed in an isolated cell, and therefrom identifying a cDNA molecule encoding a tumor-associated protein.

This invention provides a method for identifying a cDNA molecule encoding a protein associated with a cancer, comprising the steps of:

(a) immunizing an immunocompetent animal with non-tumorigenic cells transformed with a cDNA library derived from cancer cells;

(b) preparing a phage display library from splenocytes of the immunized animal;

(c) removing phage particles displaying single chain Fv and/or Fab fragments that bind to non-transformed non-tumorigenic cells from the phage display library; and

(d) identifying a cDNA molecule present in the cDNA library, wherein the protein encoded by the cDNA molecule is bound by an Fv and/or Fab fragment displayed by a phage particle in the phage display library, and therefrom identifying a cDNA encoding a protein associated with the cancer.

### BRIEF DESCRIPTION OF THE FIGURES

FIG. 1 A schematic diagram depicting a representative protocol for cDNA Antigen Targeting (CAT).

FIG. 2 A schematic diagram depicting a representative protocol for Subtraction cDNA Antigen Targeting (SCAT).

FIG. 3 A schematic diagram depicting a representative protocol for Internal cDNA Antigen Targeting (InCAT).

FIG. 4 A schematic diagram depicting a representative protocol for Subtraction Internal cDNA Antigen Targeting (SinCAT).

FIG. 5 A schematic diagram depicting a representative protocol for Tumor Rapid Expression Cloning System (T-RExCS).

FIG. 6 A schematic diagram depicting a representative protocol for Tumor Subtraction Rapid Expression Cloning System (TS-RExCS).

FIG. 7 Photographs showing untransfected CREF-Trans 6 cells.

FIG. 8 Photographs showing CREF-Trans 6 cells transfected with a human prostate carcinoma cDNA library. Cells are growing from an explanted nude mouse tumor. These cells were used for the plasmid rescue studies described herein.

FIG. 9 Depicts the partial sequence of a prostate tumor-associated polynucleotide referred to herein as PTresu-1.

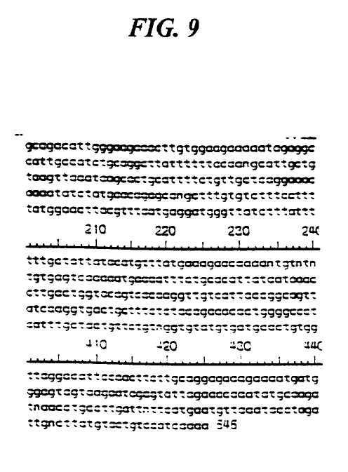

FIG. 10 Depicts the partial sequence of a prostate tumor-associated polynucleotide referred to herein as PTresu-2.

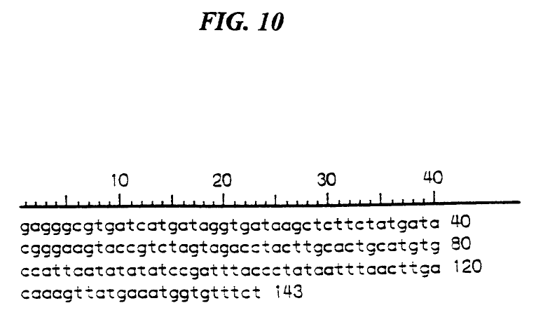

### DETAILED DESCRIPTION OF THE INVENTION

This invention provides a method for preparing antibodies directed against a protein associated with a cancer, comprising the steps of:

(a) transfecting non-tumorigenic cells with a cDNA library comprising cDNA molecules derived from mRNA of cancer cells;

(b) masking the transfected cells with antibodies raised against non-tumorigenic cells transfected with a cDNA library derived from normal cells; and

(c) generating antibodies against the masked cells, and thereby generating antibodies directed against a protein associated with a cancer.

This invention provides the above method, wherein the non-tumorigenic cells are CREF-Trans 6 cells.

This invention provides the above method, wherein the cDNA library comprising cDNA molecules derived from mRNA of cancer cells is a subtracted cDNA library.

This invention provides the above method, wherein the cDNA library comprising cDNA molecules derived from mRNA of normal cells is a subtracted cDNA library.

This invention provides the above method, wherein the cDNA molecules are operably linked to an expression vector that allows surface expression of proteins encoded by the cDNA molecules.

This invention provides the above method, wherein the cancer includes but is not limited to prostate cancer, breast cancer, lung cancer, colorectal cancer, gastric cancer, ovarian cancer, pancreatic cancer, melanoma, glioblastoma, lymphoma and leukemia.

This invention provides the above method, wherein the cancer cells are tumor cells.

This invention provides a method for identifying a cDNA molecule encoding a protein associated with a cancer, comprising the steps of:

(a) transfecting non-tumorigenic cells with a cDNA library comprising cDNA molecules derived from mRNA of cancer cells;

(b) masking the transfected cells with antibodies raised against non-tumorigenic cells transfected with a cDNA library derived form normal cells;

(c) Generating monoclonal antibodies against antigens present on the surface of the masked cells;

(d) preparing a phage display library comprising phage particles that display single chain Fv and/or Fab fragments that bind to antigens present on the surface of the masked cells; and

(e) identifying a cDNA molecule present within the cDNA library derived from cancer cells, wherein the cDNA molecule encodes an antigen that binds to a single chain Fv or Fab fragment present in the phage display library, and therefrom identifying a cDNA molecule encoding a protein associated with a cancer.

This invention provides the above method, wherein the non-tumorigenic cells are CREF-Trans 6 cells.

This invention provides the above method, wherein the cDNA library comprising cDNA molecules derived from mRNA of cancer cells is a subtracted cDNA library.

This invention provides the above method, wherein the cDNA library comprising cDNA molecules derived from mRNA of normal cells is a subtracted cDNA library.

This invention provides the above method, wherein the cDNA molecules are operably linked to an expression vector that allows surface expression of proteins encoded by the cDNA molecules.

This invention provides the above method, wherein the cancer includes but is not limited to prostate cancer, breast cancer, lung cancer, colorectal cancer, gastric cancer, ovarian cancer, pancreatic cancer, melanoma, glioblastoma, lymphoma and leukemia.

This invention provides the above method, wherein the cancer cells are tumor cells.

This invention provides a method for identifying a cDNA molecule encoding a tumor-associated protein, comprising the steps of:

(a) transfecting non-tumorigenic cells with a cDNA library derived from tumor cells;

(b) injecting the transfected cells into an immunocompromised animal;

(c) explanting any tumors that develop in the animal;

(d) isolating cells that grow from the explanted tumors; and

(e) identifying a cDNA molecule expressed in an isolated cell, and therefrom identifying a cDNA molecule encoding a tumor-associated protein.

This invention provides the above method, wherein the non-tumorigenic cells are CREF-Trans 6 cells.

This invention provides the above method, wherein the cDNA library is a subtracted library.

This invention provides the above method, wherein the tumor cells include but are not limited to prostate, breast, lung, colorectal, gastric, ovarian and pancreatic cells.

This invention provides the above method, wherein the tumor cells include but are not limited to melanoma, glioblastoma and lymphoma cells.

This invention provides the above method, wherein the immunocompromised animal includes but is not limited to nude mice, SCID mice and XID mice.

This invention provides a method for identifying a cDNA molecule encoding a protein associated with a cancer, comprising the steps of:

(a) immunizing an immunocompetent animal with non-tumorigenic cells transformed with a cDNA library derived from cancer cells;

(b) preparing a phage display library from splenocytes of the immunized animal;

(c) removing phage particles displaying single chain Fv and/or Fab fragments that bind to non-transformed non-tumorigenic cells from the phage display library; and

(d) identifying a cDNA molecule present in the cDNA library, wherein the protein encoded by the cDNA molecule is bound by an Fv and/or Fab fragment displayed by a phage particle in the phage display library, and therefrom identifying a cDNA encoding a protein associated with the cancer.

This invention provides the above method, wherein the non-tumorigenic cells are CREF-Trans 6 cells.

This invention provides the above method of claim 21, wherein the cDNA library is a subtracted cDNA library.

This invention provides the above method, wherein the cDNA molecules are operably linked to an expression vector that allows surface expression of proteins encoded by the cDNA molecules.

This invention provides the above method, wherein the cancer includes but is not limited to prostate cancer, breast cancer, lung cancer, colorectal cancer, gastric cancer, ovarian cancer, pancreatic cancer, melanoma, glioblastoma, lymphoma and leukemia.

This invention provides the above method, wherein the cancer cells are tumor cells.

As noted above, the present invention provides methods for identifying genes and proteins associated with specific disease states that involve cell growth and/or differentiation. Such disease states include cancer, as well as other conditions that involve differentiation, apoptosis, growth arrest, senescence and/or development. A protein associated with such a disease state is any protein whose expression is altered in cells affected by the disease. For example, a protein associated with a cancer (a tumor-associated protein) is any protein present in cancer cells at a higher or lower level, or in an altered form, relative to normal cells. Tumor-associated proteins include oncogenes, tumor-associated antigens and other proteins whose expression and/or activity is increased as a consequence of cancer. Cancer cells are cells that are altered as a result of a cancer. Suitable cancer cells include any tumor cells, such as tumor cell lines, primary cancer cells or tissue derived from primary or metastatic cancers. Cancer cells may be derived from any cancer including, but not limited to, prostate, breast, lung (small cell lung carcinoma (SCLC) and non-SCLC), colorectal, gastric, ovarian and pancreatic cancers, as well as melanomas, glioblastomas, lymphomas and leukemias. For each cancer cell, a suitable normal cell for comparison purposes is a cell of the same cell type as the cancer cell, but that is unaffected by the particular cancer. Normal cells may, for example, be obtained from an unaffected portion of an afflicted mammal, from a separate healthy mammal of the same species or from a cell culture. Normal cells need not be free of other conditions or diseases, provided that they are free of detectable alteration due to the disease state of interest.

Within each of the methods provided herein, nucleic acid libraries form diseased and normal cells are employed. Such libraries may be commercially available, or may be prepared using standard techniques (see Sambrook et al., Molecular Cloning: A laboratory Manual, Cold Spring Harbor Laboratories, Cold Spring Harbor, N.Y., 1989, and references cited therein). Within certain embodiments, the use of cDNA expression libraries is preferred. In addition, or alternatively, a library may be subtracted using well known subtractive hybridization techniques. For use within the subject methods, libraries are transformed or transfected into a suitable cell that is free of the disease of interest. For example, for the identification of a tumor-associated protein, a non-tumorigenic cell should be employed. Preferred non-tumorigenic cells include CREF-Trans 6 cells (ATCC Accession Number CRL 10584).

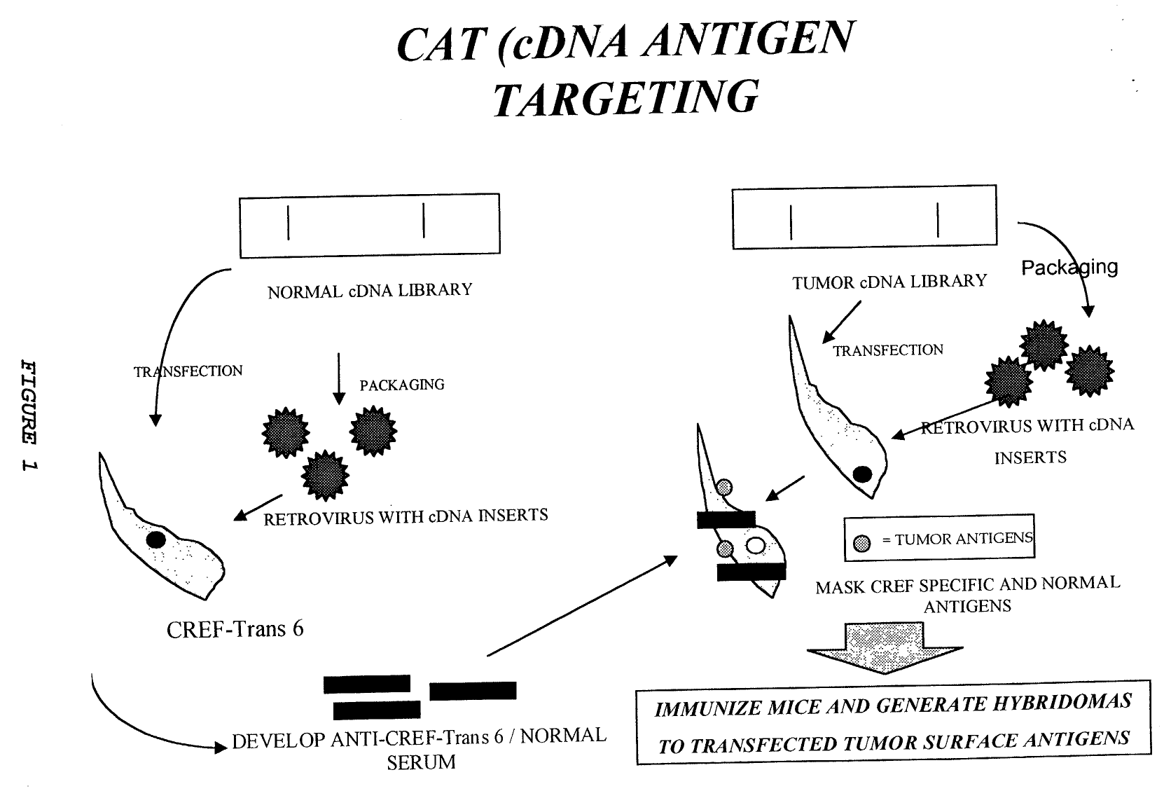

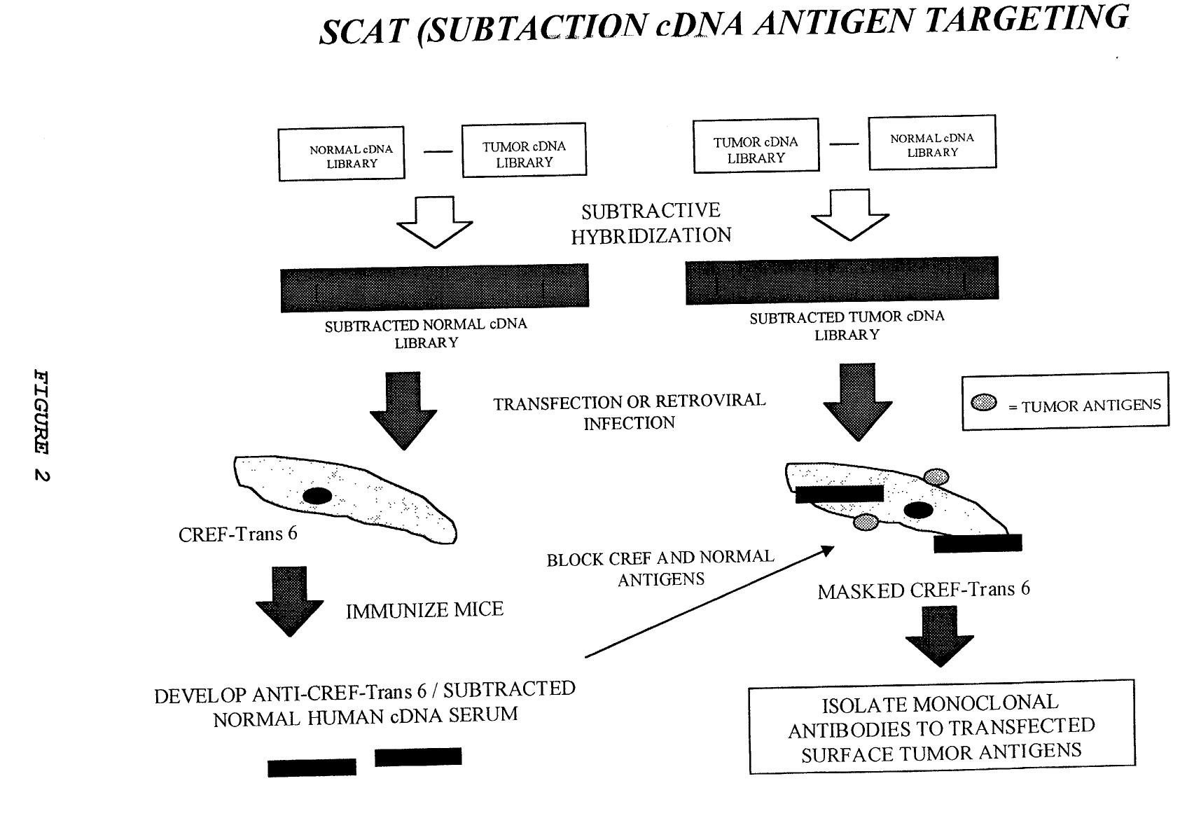

Within certain aspects, disease-associated proteins may be identified using monoclonal antibodies that react with proteins that are expressed at different levels on the surface of diseased cells, relative to normal cells. Such monoclonal antibodies may be prepared using the techniques of cDNA Antigen Targeting (CAT) and Subtraction cDNA Antigen Targeting (SCAT). The basic protocols are depicted in FIGS. 1 and 2, using the identification of tumor-associated antigens as a representative example. Briefly, cDNA libraries are derived (i.e., prepared) from mRNA of normal and cancer cells, using standard techniques. For CAT, the libraries are used without subtraction. For SCAT, the normal cDNA library is first subtracted with the library prepared from diseased cells, and the diseased cDNA library is first subtracted with the library prepared from normal cells. With or without subtraction, the cDNA library prepared from normal cells is transformed or transfected into non-tumorigenic cells as described above using any standard technique appropriate for the particular cell type including, but not limited to, retroviral infection.

Cells transfected with the normal cDNA library are then harvested at various times (e.g., 1, 2, 3 and 4 days following transfection), fixed (e.g., with 1% neutral buffered formalin) and pooled, and about 1×10⁶ cells are injected into an immunocompetent animal. An immunocompetent animal is any non-human animal whose ability to mount an immune response is unimpaired. Preferred immunocompetent animals are mice (such as Balb/c mice) rats, hamsters, guinea pigs and rabbits. Several immunizations may be employed to ensure the development of a high titer antiserum. Any suitable assay, such as an ELISA, may be used to determine the titer of the antiserum. Briefly, within one such assay, CREF-Trans 6 cells transfected with a normal cDNA library may be grown in 96 well microtiter plates until confluent. Medium is then removed, the plates washed once with PBS, and the cells fixed with 10% neutral buffered formalin for 15-20 minutes at room temperature. Wells are blocked with 3% bovine serum albumin in PBS (P-BSA) for one hour at 37° C. After washing with distilled water, goat anti-mouse IgG conjugated to horse radish peroxidase may be added to the wells for an additional hour at 37° C. After washing, TMB reagent (Kirkegaard and Perry, Gaithersburg, Md.) is added for 5-15 minutes, the reaction is stopped with 1 N sulfuric acid and the plates read at 450 nm in an ELISA reader. A high titer antiserum has a titer of at least 1:1000, as determined by such an assay.

The cDNA library derived from cancer cells (with or without subtraction) is inserted into a vector containing a selectable antibiotic resistance marker (e.g., neomycin, hygromycin or zeocin) and transfected into the non-tumorigenic cell as described above. After selection for antibiotic resistance, the cells are masked with the high titer antisera developed against the cells transfected with the normal cDNA. In general, masking refers to treating the cells with an excess of antisera under conditions such that antibodies are allowed to bind to antigens on the cell surface. For example, a high concentration of an antiserum (e.g., 1:50 or 1:100 dilution) may be mixed with the cells e.g., 1×10⁶) transfected with the cDNA library from diseased cells, and then incubated overnight at 4° C. on a rocker. Cells may then be fixed with, for example, 1% paraformaldehyde in phosphate-buffered saline (PBS) for 30 minutes at 4° C. Fixed cells may then be washed with PBS.

The masked cells (typically about 1×10⁶ masked cells) are then injected into an immunocompetent animal (e.g., a mouse) at various times over a 1-2 month period, resulting in the development of a high titer antiserum capable of binding to the cells transfected with the cancer cell-derived cDNA. Such an antiserum is also capable of binding to the cancer cells that were used to construct the cDNA library. The generation of a high titer antiserum may be monitored using, for example, an ELISA.

Monoclonal antibodies may then be generated using well known techniques such as, for example, the technique of Kohler and Milstein, Eur J. Immunol. 6:511-519, 1976, and improvements thereto. Briefly, immortal cell lines capable of producing antibodies having the desired specificity are prepared from spleen cells obtained from an animal immunized as described above. The spleen cells are immortalized by, for example, fusion with a myeloma cell fusion partner, preferably one that is syngeneic with the immunized animal. Single colonies are selected and their culture supernatants tested for binding activity against the modulating agent or antigenic portion thereof. Hybridomas having high reactivity and specificity are preferred.

Monoclonal antibodies may be isolated from the supernatants of growing hybridoma colonies, with or without the use of various techniques known in the art to enhance the yield. Contaminants may be removed from the antibodies by conventional techniques, such as chromatography, gel filtration, precipitation, and extraction. Antibodies having the desired activity may generally be identified using immunofluorescence analyses of tissue sections, cell or other samples where the target antigen is localized.

In addition to such monoclonal antibodies, recombinatorial phage display libraries may be prepared using well known techniques (as described, for example, by McCafferty et al, Nature 348:552-554, 1990; Barbas et al., Proc. Natl. Acad Sci. USA 88:7879-7982, 1991) Such libraries contain phage particles that display single chain Fv and Fab fragments that specifically bind to antigens expressed on the surface of the diseased cells. Phage display libraries may generally be used to expression clone genes encoding disease-associated antigens using well known techniques.

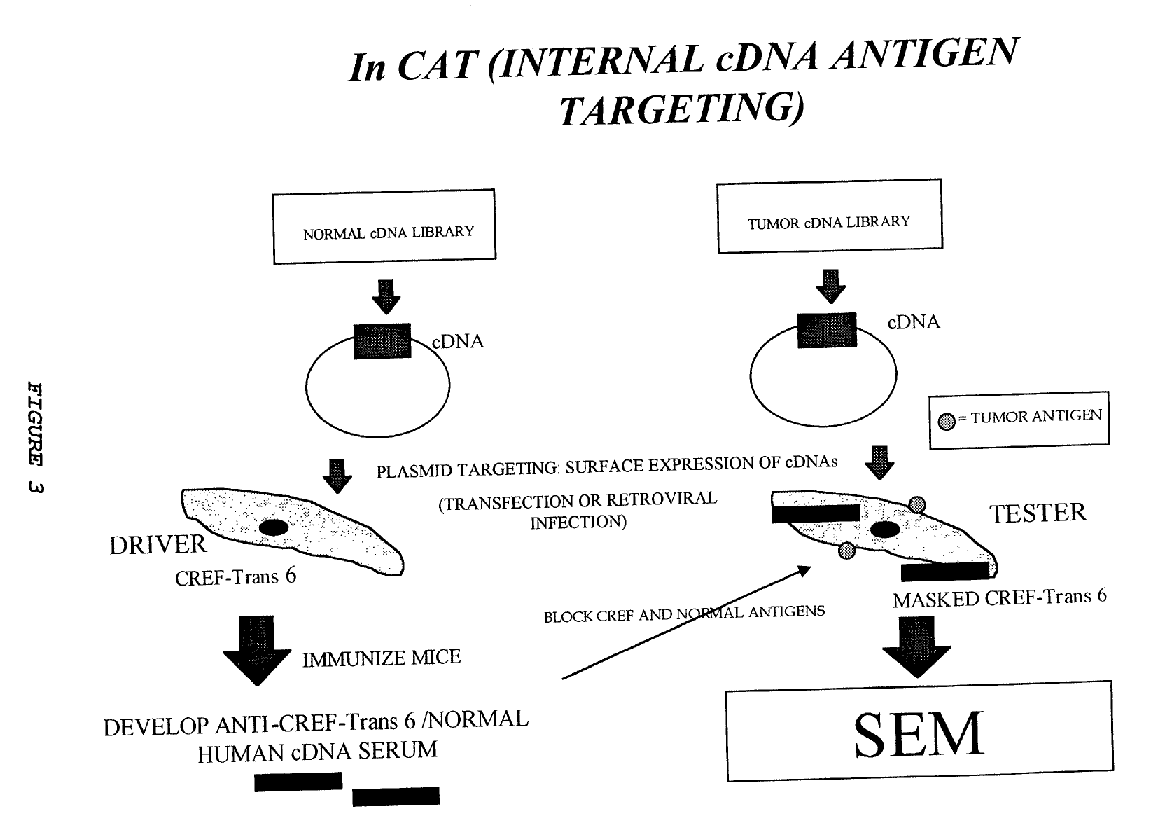

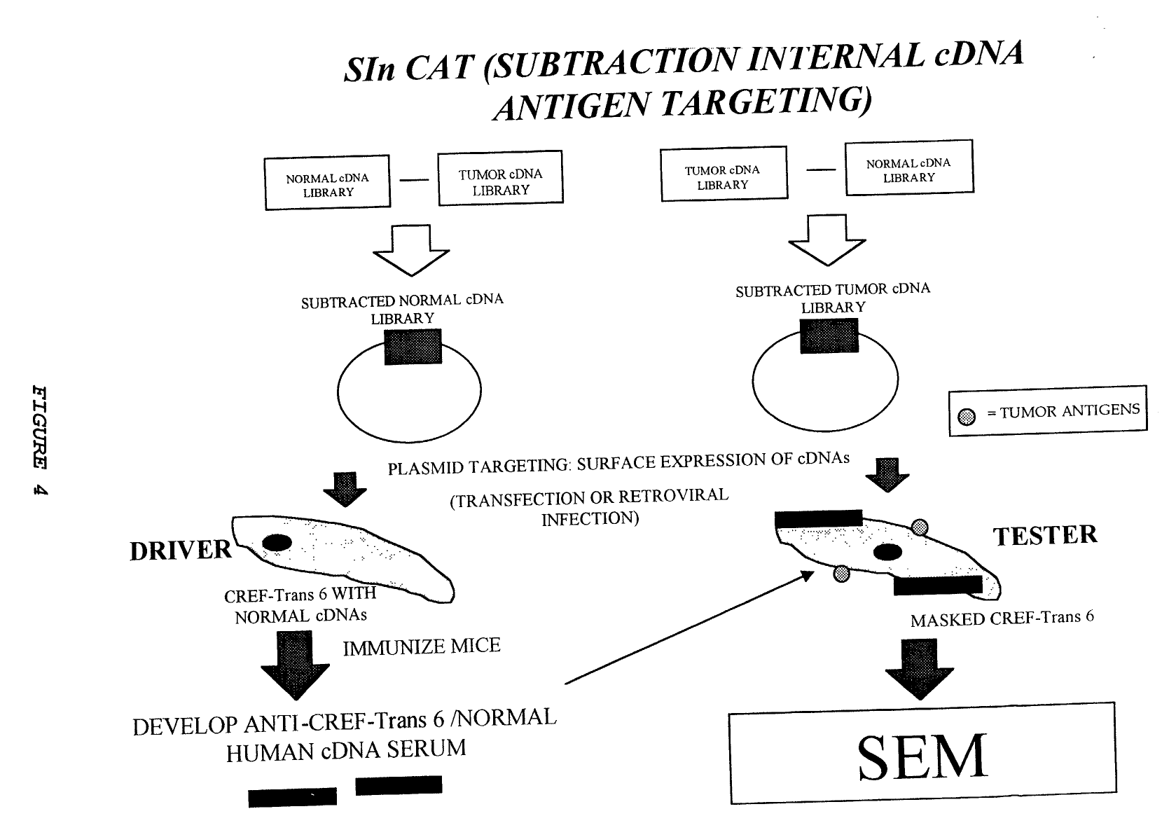

Within related aspects, internal (i.e., not typically presented on the cell surface) disease-associated proteins may be identified using techniques referred to herein as Internal cDNA Antigen Targeting (INCAT) and Subtraction Internal cDNA Antigen Targeting (SInCAT). These techniques (depicted in FIGS. 3 and 4 for a representative procedure designed to identify tumor-associated antigens) are designed to present internal antigens (including nuclear and/or cytoplasmic proteins) that are associated with a cancer on the surface of a cell, so as to permit the generation of monoclonal antibodies that bind to such antigens. Briefly, InCAT and SInCAT are performed as described above for CAT and SCAT, except that the cDNA libraries are cloned into a surface targeting vector that allows surface expression of cDNA gene products that are normally expressed in the nucleus or cytosol. For example, the vector pDisplay (available from Invitrogen) may be employed.

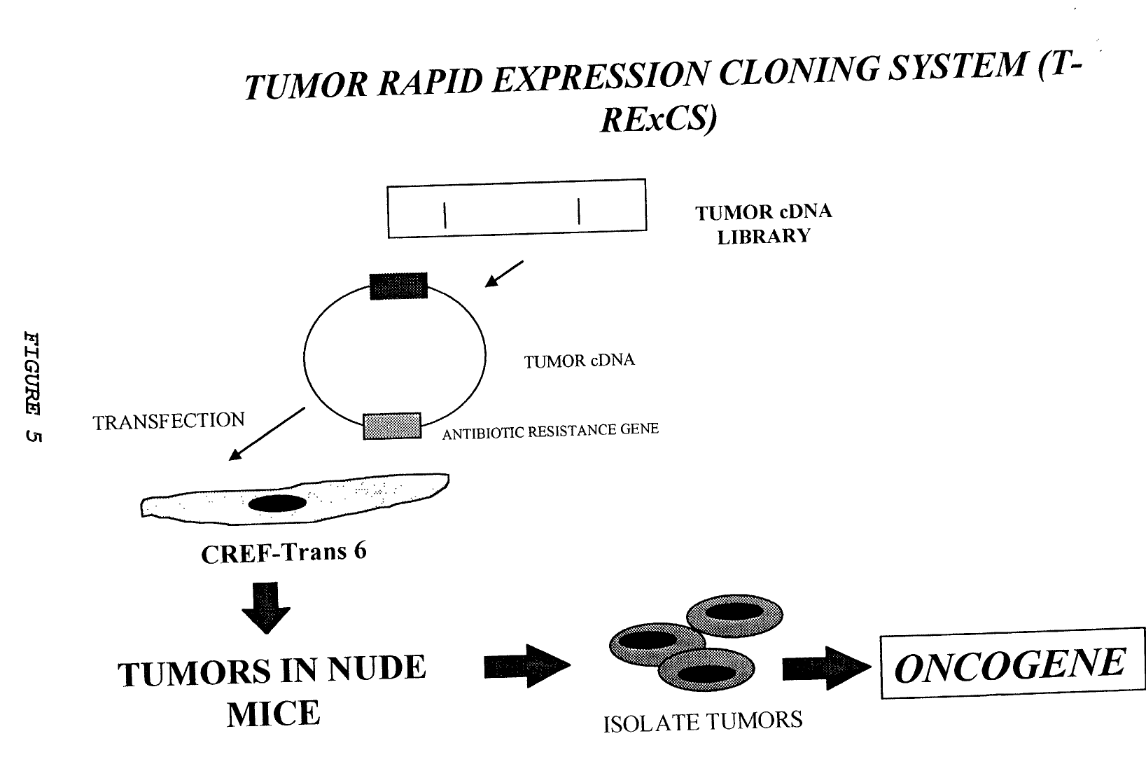

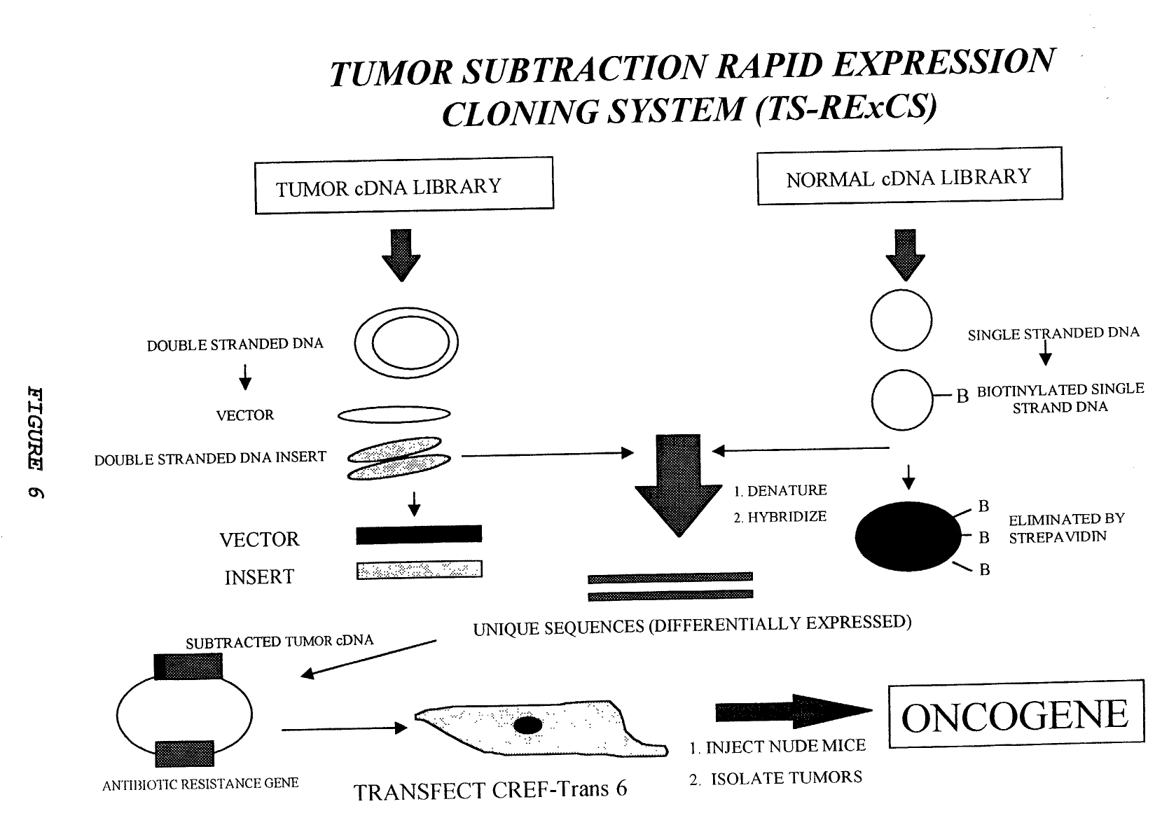

Within other aspects, cDNA molecules encoding tumor-associated antigens may be identified using techniques referred to herein as Tumor Rapid Expression Cloning System (T-RExCS) and Tumor Subtraction Rapid Expression Cloning System (TS-RExCS). Representative embodiments of these methods are provided within FIGS. 5 and 6.

Briefly, such methods comprise the steps of (1) transfecting tumor-derived cDNA expression libraries (prepared in a vector containing selectable antibiotic resistance from cell lines or from primary or metastatic cancer tissues) into non-tumorigenic cells; (2) injecting pooled colonies into immunocompromised animals (i.e., non-human animals whose ability to mount an immune response is detectably impaired) such as nude mice, SCID mice, or XID mice; (3) excising any tumors that are formed and explanting the tumors in tissue culture dishes containing medium supplemented with fetal bovine serum and the selecting antibiotic; (4) isolating cells that grow from the explants (e.g., with trypsin); and (5) identifying oncogenes and tumor associated antigens from such cells by any suitable immunological or gene cloning technique, such as surface epitope mapping (SEM), phage rescue, PCR, differential display or subtraction hybridization. TS-RExCS is identical to T-RExCS except that the cDNA library is first subtracted with a normal cDNA library.

Within further aspects, cDNA molecules encoding disease-associated proteins may be identified using methods referred to herein as “Rapid Antigen Cloning” (RAC) or “Rapid Antigen Discovery” (RAD). For example, a cancer-associated protein may be identified by (1) immunizing an immunocompetent animal with non-tumorigenic cells transformed (or transfected) with nucleic acid derived form a cancer cell; (2) generating a phage display library from splenocytes of the immunized animal; (3) enriching the library for phage specific for transformed cells by removing nonspecific single chain Fvs and/or Fabs that bind to nontransformed cells; and (4) expression cloning the original transformed gene. These steps are described in further detail below.

As noted above, a non-tumorigenic cell may be transformed or transfected with a library prepared from cancer cells using any standard technique appropriate for the particular cell type including, but not limited to, retroviral infection. Several immunizations may be employed to ensure the development of an IgG response in the animal, and the effectiveness of the response may be determined using an ELISA.

Following the development of an immune response, the animals are sacrificed and their splenocytes removed. A polyclonal phage display library may then be generated using standard methods, such as the Pharmacia Recombinant Phage Antibody System (Pharmacia, Piscataway, N.J.). The phage library is then exhaustively panned versus non-transfected non-tumorigenic cells (or non-tumorigenic cells transfected with DNA from normal cells) to remove phage particles displaying single chain Fvs or Fabs that are not specific to the cancer. “Exhaustively panned,” as used herein, means that the single chain Fvs and Fabs displayed by the phage particles do not bind detectably to an irrelevant cell line or antigen. In other words, the exhaustively panned library should not bind to fixed CREF-Trans 6 cells, as measured by ELISA. Briefly, within one representative panning procedure, negative (untransfected) CREF-Trans 6 cells are harvested with 2 mM EDTA and resuspended in P-BSA at a concentration 10⁶ cells/ml. The phage antibody library (100 μl=1010-1011 cfu) is then incubated for 1 hour in 1 ml of the cell suspension at 4° C. or 37° C. 4° C. is preferred for inhibiting internalization. The phage supernatant is removed following centrifugation and added to the target cells (transfected CREF-Trans 6) and incubated for 1 hour. Cells are then washed five times with P-BSA and specific phage eluted with 100 mM glycine pH2.2 or by cell lysis. Phage may be amplified in E. Coli and subjected to further rounds of panning (see Portolano et al., J. Immunol, 135:2839-2851. 1993) until the phage particles do not bind detectably to the CREF-Trans 6 cells.

The remaining population of phage is then enriched for phage particles displaying single chain Fvs that bind to antigens expressed preferentially in the transformed cells, as well as to cells or tissues used as the source of nucleic acid. The enriched phage library may then be used to expression clone the original nucleic acid involved in the transformation process. Any of a variety of well known techniques may be used. For example, cDNA libraries in a phage expression vector may be used to infect a lawn of E. coli. Following lysis, transformed cDNAs may be blotted to nitrocellulose filter paper and probed with the enriched phage library. A positive binding event may be demonstrated using an anti-phage antibody conjugated to a reporter or to a radioactive label. Positive cDNAs may then be isolated and sequenced.

It will be apparent to those of ordinary skill in the art that the above techniques may be modified to permit the identification of proteins that are preferentially expressed in normal cells, as compared to cancer cells. For example, to identify such proteins, the transformed cells in RAC and RAD should be cancer cells, and the library should be prepared from normal cells.

Antigens associated with one or more cancers that are identified using a method as described herein may be used in a variety of ways. Such antigens may be used as markers for diagnosing and/or monitoring a disease in an animal such as a human. For example, the level of antigen may be evaluated in an appropriate sample (e.g., a tumor biopsy) obtained from a patient, and compared to the level observed in similar samples obtained from individuals that are, and are not, afflicted with a particular disease.

There are a variety of assay formats (e.g., ELISA) known to those of ordinary skill in the art for using antibodies to detect the level of antigen in a sample. See, e.g., Harlow and Lane, Antibodies: A Laboratory Manual, Cold Spring Harbor Laboratory, 1988. In one embodiment, the assay involves the use of antibody immobilized on a solid support to bind to and remove the antigen from the sample. The bound antigen may then be detected using a detection reagent that binds to the antibody/antigen complex and contains a detectable reporter group. Alternatively, a competitive assay may be utilized, in which an antigen is labeled with a reporter group and allowed to bind to the immobilized antibody after incubation with the sample. The extent to which components of the sample inhibit the binding of the labeled antigen to the antibody is indicative of the reactivity of the sample with the immobilized antibody.

A solid support for use within such assays may be any material known to those of ordinary skill in the art to which the polypeptide may be attached. For example, the support may be a test well in a microtiter plate or a nitrocellulose or other suitable membrane. Alternatively, the support may be a bead or disc, such as glass, fiberglass, latex or a plastic material such as polystyrene or polyvinylchloride. An antibody may be bound to the solid support using a variety of techniques known to those in the art, which are amply described in the patent and scientific literature. In the context of the present invention, the term “bound” refers to both noncovalent association, such as adsorption, and covalent attachment (which may be a direct linkage between the antigen and functional groups on the support or may be a linkage by way of a cross-linking agent).

Binding by adsorption to a well in a microtiter plate or to a membrane is preferred. In such cases, adsorption may be achieved by contacting the antibody, in a suitable buffer, with the solid support for a suitable amount of time. The contact time varies with temperature, but is typically between about 1 hour and 1 day.

To determine the presence or absence of antigen in a sample, the signal is generally compared to a signal that corresponds to a predetermined cut-off value. In one preferred embodiment, the cut-off value is the average mean signal obtained when the immobilized antibody is incubated with samples from a patient that is not afflicted with the disease of interest. In general, a sample generating a signal that is three standard deviations above the predetermined cut-off value is considered positive (i.e., reactive with the antibody). of course, numerous other assay protocols exist that are suitable for use with the antigens and antibodies described herein. The above descriptions are intended to be exemplary only.

Antigens identified using the methods provided herein may also be useful in the development of new therapies. In particular, surface antigens specific to cancer cells may provide novel approaches to targeting imaging, cytotoxic and/or therapeutic agents to cancer cells. For example, an agent may be linked to an antibody or fragment thereof that binds to the antigen, and administered to a patient. Internal antigens may generally be used for the identification of drugs that alter the antigen activity or pattern of expression. Antigens identified as described herein may also be used as targets for small molecule screens. For example, the downregulation of an identified gene can be monitored using Northern blot analysis of PCR following treatment of target cells with small molecule libraries. Alternatively, the promoter of an isolated gene may be identified using standard techniques, and then fused to a reporter gene (e.g., luciferase, β-galactosidase, CAT and the like) and inserted into the original cell line. The cell line expressing the reporter gene can then be used for screening a small molecule library for compounds that alter expression from the promoter.

This invention will be better understood from the Experimental Details that follow. However, one skilled in the art will readily appreciate that the specific methods and results discussed are merely illustrative of the invention as described more fully in the claims which follow thereafter.

### EXPERIMENTAL DETAILS

Preparation of Cell Lines Expressing Tumor Associated Genes

This Example illustrates the use of Tumor Rapid Expression Cloning System (T-RExCS) to prepare cell lines that express tumor associated genes.

CDNAs were prepared from mRNA isolated from prostate, breast and lung carcinomas. Such cDNA molecules were cloned into pcDNA3.1 and used to transfect CREF Trans 6 cells. CREF-Trans 6 cells were plated in 6 well microplates at a concentration of 1×10⁵ cells/well in RPMI-1640 medium supplemented with 10% FBS (growth medium). After the cells reached confluency, 10 μg of cDNA from a prostate tumor cDNA library was transfected using the Lipofectamine protocol (GIBCO BRL Life Technologies). Briefly 10 μg of cDNA was mixed with Lipofectamine reagent in serum free medium (final concentration of 1 ml) for 15-45 minutes at room temperature. The solution was layered over the cells for 4-6 hours at 37° C. Following incubation, 1 ml of medium containing 20% FBS was added and the plates maintained overnight at 37° C. Fresh growth medium was added to the wells and the cells grown for an additional 24 hours at 37° C. when medium containing 200 μg/ml of G418 (selection medium) was added. Surviving cells formed colonies which were expanded and used to inoculate nude mice.

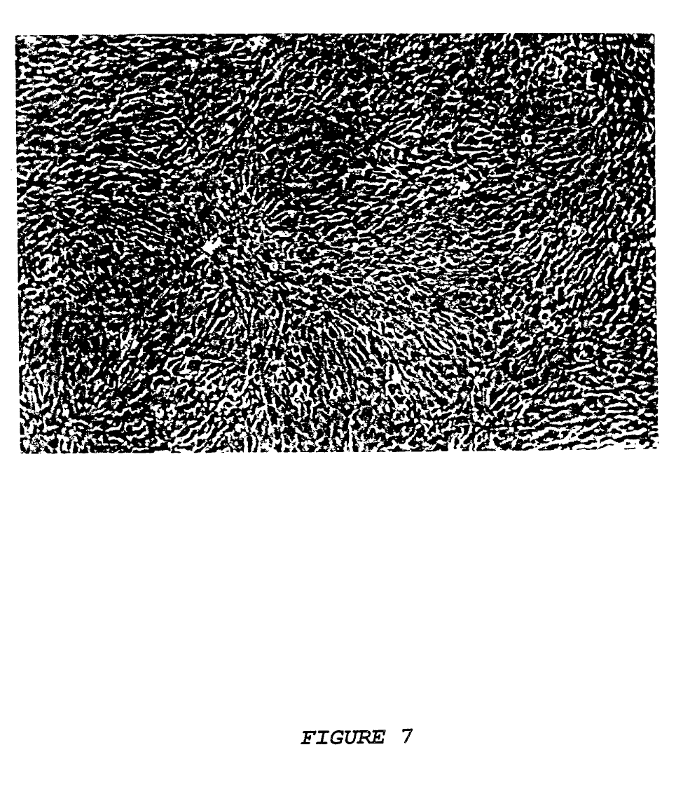

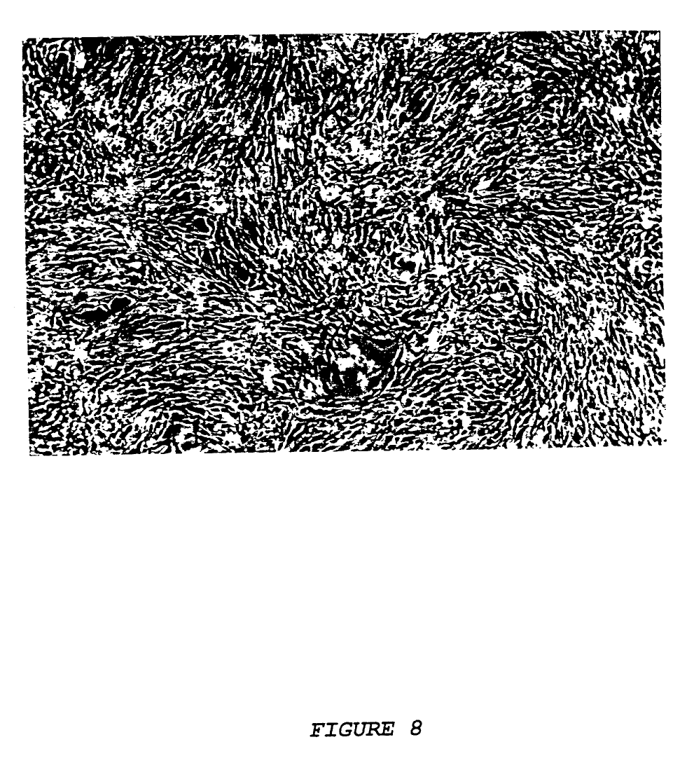

Cells were removed from growth vessels with trypsin-EDTA and one million cells injected into the flanks of nude mice (1 injection into 3 mice). Animals were observed until tumors formed (about 6-8 weeks). Animals were sacrificed by cervical dislocation and the tumors excised asceptically. Tumor tissue was cut into small (1-2 mm² sections) and allowed to adhere to the surface of 100 mm² petri dishes. After 2-3 weeks, there was an outgrowth of cells from the explants. These cells were collected, expanded and used for the plasmid rescue studies. A photograph of cells transfected with a human prostate carcinoma cDNA library and growing from an explanted nude mouse tumor is presented in FIG. 8. For comparison, the untransfected CREF-Trans 6 cells are shown in FIG. 7.

The explanted cultures were treated with G418 to eliminate contaminating murine fibroblasts. The genomic DNAs were extracted and purified from the cells. 10 μg of the genomic DNAs were digested at 37° C. overnight using SacII, NHEI, and EcoRV. The digested products were diluted ten-fold and ligated at 14° C. overnight. The ligated samples were transformed into XL1-blue competent cells at 4° C. for 30 minutes, which were treated by heat shock at 42° C. for 1 minute, and placed on ice for an additional 2 minutes. The transformed E. coli cells were then spread on LB agar plates containing ampicillin and incubated overnight at 37° C.

After incubation, only digested genomic DNAs were extracted from the primary tumor cells forming colonies. Forty colonies were picked and cultured for use in a mini-prep. Here, the plasmid DNAs, extracted from the colonies, were digested using NHEI for two hours at 37° C. and the digested products were separated electrophoretically using a 1% agarose gel. The different insert sizes were sequenced, and novelty was confirmed based on a search of public data bases. Two novel genes were isolated: PT rescue #1 (SEQ ID NO:1) and PT rescue #2 (SEQ ID NO:2).

Gene expression patterns were done using a human multiple tissue Northern blot (Clontech) and a human multiple tumor cell line blot. The PT rescue #1 gene is expressed in all tissues and cell lines and is highly expressed in skeletal muscle and heart tissue. This gene has a >10 fold higher expression pattern in several tumor cell lines compared to normal tissue and cell lines.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is

1. A method for preparing antibodies directed against a protein associated with a cancer, comprising the steps of:
(a) transfecting non-tumorigenic cells with a cDNA library comprising cDNA molecules derived from mRNA of cancer cells;
(b) masking the transfected cells with antibodies raised against non-tumorigenic cells transfected with a cDNA library derived from normal cells; and
(c) generating antibodies against the masked cells, and thereby generating antibodies directed against a protein associated with a cancer.

2. The method of claim 1, wherein the non-tumorigenic cells are CREF-Trans 6 cells.

3. The method of claim 1, wherein the cDNA library comprising cDNA molecules derived from mRNA of cancer cells is a subtracted cDNA library.

4. The method of claim 1, wherein the cDNA library comprising cDNA molecules derived from mRNA of normal cells is a subtracted cDNA library.

5. The method of claim 1, wherein the cDNA molecules are operably linked to an expression vector that allows surface expression of proteins encoded by the cDNA molecules.

6. The method of claim 1, wherein the cancer is selected from the group consisting of prostate cancer, breast cancer, lung cancer, colorectal cancer, gastric cancer, ovarian cancer, pancreatic cancer, melanoma, glioblastoma, lymphoma and leukemia.

7. The method of claim 1, wherein the cancer cells are tumor cells.

8. A method for identifying a cDNA molecule encoding a protein associated with a cancer, comprising the steps of:
(a) transfecting non-tumorigenic cells with a cDNA library comprising cDNA molecules derived from mRNA of cancer cells;
(b) masking the transfected cells with antibodies raised against non-tumorigenic cells transfected with a cDNA library derived form normal cells;
(c) Generating monoclonal antibodies against antigens present on the surface of the masked cells;
(d) preparing a phage display library comprising phage particles that display single chain Fv and/or Fab fragments that bind to antigens present on the surface of the masked cells; and
(e) identifying a cDNA molecule present within the cDNA library derived from cancer cells, wherein the cDNA molecule encodes an antigen that binds to a single chain Fv or Fab fragment present in the phage display library, and therefrom identifying a cDNA molecule encoding a protein associated with a cancer.

9. The method of claim 8, wherein the non-tumorigenic cells are CREF-Trans 6 cells.

10. The method of claim 8, wherein the cDNA library comprising cDNA molecules drived from mRNA of cancer cells is a subtracted cDNA library.

11. The method of claim 8, wherein the cDNA library comprising cDNA molecules derived from mRNA of normal cells is a subtracted cDNA library.

12. The method of claim 8, wherein the cDNA molecules are operably linked to an expression vector that allows surface expression of proteins encoded by the cDNA molecules.

13. The method of claim 8, wherein the cancer is selected from the group consisting of prostate cancer, breast cancer, lung cancer, colorectal cancer, gastric cancer, ovarian cancer, pancreatic cancer, melanoma, glioblastoma, lymphoma and leukemia.

14. The method of claim 8, wherein the cancer cells are tumor cells.

15. A method for identifying a cDNA molecule encoding a tumor-associated protein, comprising the steps of:
(a) transfecting non-tumorigenic cells with a cDNA library derived from tumor cells;
(b) injecting the transfected cells into an immunocompromised animal;
(c) explanting any tumors that develop in the animal;
(d) isolating cells that grow from the explanted tumors; and
(e) identifying a cDNA molecule expressed in an isolated cell, and therefrom identifying a cDNA molecule encoding a tumor-associated protein.

16. The method of claim 15, wherein the non-tumorigenic cells are CREF-Trans 6 cells.

17. The method of claim 15, wherein the cDNA library is a subtracted library.

18. The method of claim 15, wherein the tumor cells are selected from the group consisting of prostate, breast, lung, colorectal, gastric, ovarian and pancreatic cells.

19. The method of claim 15, wherein the tumor cells are selected from the group consisting of melanoma, glioblastoma and lymphoma cells.

20. The method of claim 15, wherein the immunocompromised animal is selected from the group consisting of nude mice, SCID mice and XID mice.

21. A method for identifying a cDNA molecule encoding a protein associated with a cancer, comprising the steps of:
(a) immunizing an immunocompetent animal with non-tumorigenic cells transformed with a cDNA library derived from cancer cells;
(b) preparing a phage display library from splenocytes of the immunized animal;
(c) removing phage particles displaying single chain Fv and/or Fab fragments that bind to non-transformed non-tumorigenic cells from the phage display library; and
(d) identifying a cDNA molecule present in the cDNA library, wherein the protein encoded by the cDNA molecule is bound by an Fv and/or Fab fragment displayed by a phage particle in the phage display library, and therefrom identifying a cDNA encoding a protein associated with the cancer.

22. The method of claim 21, wherein the non-tumorigenic cells are CREF-Trans 6 cells.

23. The method of claim 21, wherein the cDNA library is a subtracted cDNA library.

24. The method of claim 21, wherein the cDNA molecules are operably linked to an expression vector that allows surface expression of proteins encoded by the cDNA moleucles.

25. The method of claim 21, wherein the cancer is selected from the group consisting of prostate cancer, breast cancer, lung cancer, colorectal cancer, gastric cancer, ovarian cancer, pancreatic cancer, melanoma, glioblastoma, lymphoma and leukemia.

26. The method of claim 21, wherein the cancer cells are tumor cells.


SEQUENCE LISTING (2 sequences)

SEQ ID NO: 1 - DNA, 545 bp, homo sapiens
  misc_feature (71)...(71): n = A,T,C or G
gcagacattgggaagaaacttgtggaagaaaaatagaggccattgccatctgcaggcttattttttacaangcattgctgtaagttaaataagcactgcattttctgttgctcaggaaacaaaatatctatgaacagagcangctttgtgtctttccttttatggaacttacgtttcatgaggatgggttatctttattttttgctattatacatgtttatgaaagaacaaaantgtntntgtgagtcaaaaatgaaaatttctgcacattataataaaccttgactggtacagtcacaaggttgtcattaccggcagttatcaaggtgactgctttctctacagaaacactggggccctcatttgctactgttctgtnggtgtatgtgatgcactgtggttaggacattccaacttcttgcaggagaaagaaaatgatgggagtagtaagaatagcgtattagaaacaaatatgcaagatnaacctgccttgattnttcatgaatgttaaatacctagattgncttatgtactgtccatcaaaa

SEQ ID NO: 2 - DNA, 143 bp, homo sapiens
gagggcgtgatcatgataggtgataagctcttctatgatacgggaagtaccgtctagtagacctacttgcactgcatgtgccattaatatatatccgatttaccctataatttaacttgacaaagttatgaaatggtgtttct